(19) United States
(12) Patent Application Publication
(10) Pub. No.: US 2024/0215625 A1
(43) Pub. Date: Jul. 4, 2024

(54) POROUS STARCH AS SPRAY-DRYING AID IN THE PREPARATION OF FLAVOR POWDERS

(71) Applicant: ROQUETTE FRERES, LESTREM (FR)

(72) Inventors: Bernard PORA, Shanghai (CN); Jovin HASJIM, BETHUNE (FR); Shiyao YU, SHANGHAI (CN); Jie SUN, SHANGHAI (CN)

(21) Appl. No.: 17/907,175

(22) PCT Filed: Mar. 18, 2021
(86) PCT No.: PCT/EP2021/056894

§ 371 Date: Sep. 23, 2022

(30) Foreign Application Priority Data
Mar. 26, 2020 (CN) 202010225793.2

(51) Int. Cl. A23L 29/212; A23L 3/46; A23L 27/50; C12N 9/26; C12P 19/04; C12P 19/14

(52) U.S. Cl. CPC A23L 29/212; A23L 3/46; A23L 27/50; C12N 9/2414; C12P 19/04; C12P 19/14; C12Y 302/01001

(57) ABSTRACT

The present invention relates to the use of porous starch as spray-drying aid in the preparation of a flavor powder. The present invention also relates to a process of fabricating the flavor powder and to a flavor powder comprising porous starch obtained from said process. Also, the present invention relates to a flavor powder comprising porous starch as spray-drying aid.

### FIELD OF THE INVENTION

The present invention relates to the use of porous starch as spray-drying aid in the preparation of a flavor powder. The present invention also relates to a process of fabricating the flavor powder and to a flavor powder comprising porous starch obtained from said process. Also, the present invention relates to a flavor powder comprising porous starch as spray-drying aid.

### BACKGROUND

Traditional processes to make the flavor powder use dextrin or maltodextrin as carrier and/or spray-drying aid. Spray drying is the most common method in the industry to produce powdered ingredients/foods/beverages due to its rapid drying, preventing the off flavor caused by the maillard reaction or overheating. Without dextrin or maltodextrin the flavoring molecules are small and difficult to be dried into powder form due to the high hygroscopicity. Therefore, it is important to have drying aid, such as dextrins and maltodextrins, to reduce the hygroscopicity in order to obtain a powder form and to maintain the powder form upon storage. However, consumers who have become increasingly reluctant to purchase products with a list of chemical substances on the label or chemically modified ingredients also have a bad perception of dextrins and maltodextrins.

There is thus a need to provide another spray-drying aid, more generally drying-aid, which can be classified as a clean label ingredient to replace dextrins and maltodextrins in the preparation of flavor powders.

The present inventors have surprisingly found that a specific porous starch can be used as spray-drying aid in the preparation of flavor powders.

### SUMMARY OF THE INVENTION

A first object of the present invention is directed to the use of porous starch as spray-drying aid in the preparation of a flavor powder containing no contain maltodextrin and no dextrin.

A second object of the present invention relates to process of fabricating a flavor powder comprising a step of adding porous starch as spray-drying aid, wherein said process does not comprise a step of adding maltodextrin and/or dextrin.

A third object of the present invention relates to a flavor powder comprising porous starch obtained from the process as defined in the present invention.

A fourth object of the present invention relates to a flavor powder comprising a spray-drying aid containing or consisting of porous starch, wherein said flavor powder does not contain dextrin or maltodextrin.

### DETAILED DESCRIPTION

A first object of the present invention relates to the use of porous starch as spray-drying aid in the preparation of a flavor powder containing no maltodextrin and no dextrin.

In the present invention, “flavor powder” refers to a flavoring or seasoning powder obtained by spray-drying a flavoring solution or seasoning solution (including extract, sauce, emulsion and suspension). A flavor powder is convenient for transport, dry mixing and storing, and it may have longer shelf-life and can be more resistant to heat than the corresponding flavoring solution or seasoning solution.

The flavor powder of the present invention can be used in dry mixes for example in the flavorings or seasonings for noodles, rice, puffed foods, snacks, biscuits, beverages and soups. It can also be used for mixing in dough or batter for snacks and bakery products.

In a preferred embodiment of the present invention, the flavor powder is a bouillon powder, a seasoning powder, a seed extract powder, a leaf or vegetable extract powder, a fruit extract powder, a mushroom extract powder, a yeast extract powder, a miso powder, a soy sauce powder, an artificial or synthetic flavor powder and mixtures thereof, and preferably a soy sauce powder.

As used herein, the expression “spray-drying aid” refers to a compound that is used to reduce the stickiness or hygroscopicity of a powder by increasing the amount of larger molecules. Without spray-drying aid, small molecules form powder particles that stick together and stick to the walls of the dryer, causing operational problems and low production yield. The spray-drying aid also increases the glass transition temperature of the mixture. Glass transition temperature (Tg) is a temperature at which the transition in a solid amorphous material occurs from hard, solid, brittle state into soft, rubbery, elastic state as the temperature increases. Without spray-drying aid, when the spray drying temperature is higher than Tg, the small molecules have high molecular mobility and tend to form soft particles with sticky surface, and as the results, they turn in a paste-like structure instead of a powder material. Thus, high-molecular weight spray-drying aid is necessary to increase the Tg of the food system, and in turn to minimize the stickiness problem of the particles.

More generally, in the present invention, the porous starch may also be used as a carrier, microencapsulation-aid, or drying-aid such as freeze-drying aid or spray-drying aid, in the preparation of a flavor powder.

As used herein the expression “porous starch” refers to a granular native starch that has been hydrolyzed by one or multiple amylolytic enzymes until multiple pores are visible surface of the starch on the granules by microscopic technique.

As used herein the expression “native starch” refers to a starch coming from natural sources. It does not result from enzymatic or chemical processing methods. Typical native sources for the starches are cereal, tubers, roots, legumes and fruits. In the present invention, native starch may be recovered from native sources such as wheat, waxy wheat, maize, waxy maize, rice, waxy rice, tapioca, waxy tapioca, potato, waxy potato, sweet potato, waxy sweet potato, pea, mung bean, millet, sago, sorghum, quinoa, arrowroot, amaranth, lotus root and buckwheat by extraction processes. Native starch is normally extracted using either wet milling or dry milling known processes.

An example of a first starch extraction process comprises the following steps:
1) cleaning of grain kernels from foreign matters;
2) steeping of the grain in water, alkaline solution or a solution containing a reducing agent to soften the kernels and to facilitate the separation of starch and protein;
3) optionally, coarse grinding followed by hydrocyclone to remove the germ from the kernel;
4) fine grinding of the remaining grain kernel to release the fiber, protein, and starch;
5) passing through screens with various opening sizes to separate fiber from protein and starch;
6) optionally, removing the excess water in slurry containing starch and protein;
7) separating protein from starch by density, such as using multiple-stage hydrocyclone;
8) drying the starch, such as using centrifugal filter, vacuum filter, belt-type dryer, and/or flash dryer;
9) recovering the dried starch.

Another example of a second starch extraction process comprises the following steps:
1) cleaning and washing of starchy root or tuber from dirt and sticks;
2) removing the peel of the starchy root or tuber and chopping the flesh into chunks;
3) pulverizing the roots into pulpy slurry;
4) removing the coarse and fine fiber from starch slurry by screens and/or filter cloths with large and fine opening sizes;
5) concentrating starch slurry using two- or three-phase separator or a series of hydrocylone;
6) dewatering the starch using centrifuge, high-pressure filtration or press filter;
7) drying the starch using flash dryer;
8) recovering the dried starch.

Advantageously, the extraction process is free of organic solvents and free of chemical reactants and there is no chemical transformation.

The native starch used in the present invention for the preparation of porous starch may be wheat starch, waxy wheat starch, maize starch, waxy maize starch, rice starch, waxy rice starch, tapioca starch, waxy tapioca starch, potato starch, waxy potato starch, sweet potato starch, waxy sweet potato starch, pea starch, mung bean starch, millet starch, sago starch, sorghum starch, quinoa starch, arrowroot starch, amaranth starch, lotus root starch, buckwheat starch, and mixtures thereof.

Thus, in a specific embodiment of the present invention, the porous starch is selected from the group consisting of porous wheat starch, porous waxy wheat starch, porous maize starch, porous waxy maize starch, porous rice starch, porous waxy rice starch, porous tapioca starch, porous waxy tapioca starch, porous potato starch, porous waxy potato starch, porous sweet potato starch, porous waxy sweet potato starch, porous pea starch, porous mung bean starch, porous millet starch, porous sago starch, porous sorghum starch, porous quinoa starch, porous arrowroot starch, porous amaranth starch, porous lotus root starch, porous buckwheat starch, and mixtures thereof.

Preferably, the native starch used in the present invention for the preparation of porous starch may be an A-type crystalline starch such as wheat starch, waxy wheat starch, maize starch, waxy maize starch, rice starch, waxy rice starch, tapioca starch, waxy tapioca starch and mixtures thereof, preferably rice starch and waxy rice starch.

Thus, in a preferred embodiment of the present invention, the porous starch is selected from the group consisting of porous wheat starch, porous waxy wheat starch, porous maize starch, porous waxy maize starch, porous rice starch, porous waxy rice starch, porous tapioca starch, porous waxy tapioca starch and mixtures thereof, preferably porous rice starch and porous waxy rice starch.

According to the present invention, the porous starch may be produced through an enzyme hydrolysis of native starch granules with one or multiple amylolytic enzymes, such as alpha-amylase and amyloglucosidase, at a temperature inferior to the gelatinization temperature of the starch. The main enzymatic reaction is hydrolysis and there are no substitution, oxidation and reduction reactions, such as the introduction of new ester and ether groups and the conversion of hydroxyl groups to carbonyl and carboxyl groups.

In a preferred embodiment of the present invention, the enzyme hydrolysis enables to provide a porous starch with a low viscosity upon gelatinization, similar to that of dextrin and maltodextrin.

In a preferred embodiment of the present invention, alkaline pH or alcohols solutions are not used to produce clean label starch and acid and base solutions are only used as processing aids to adjust the pH for enzyme hydrolysis and enzyme deactivation. Thus, in a preferred embodiment of the present invention, porous starch is not obtained by acid hydrolysis.

In a preferred embodiment of the present invention, the porous starch is obtained from native starch granules by enzyme hydrolysis only and preferably by enzyme hydrolysis using alpha-amylase.

Advantageously, the porous starch used in the present invention in the preparation of a flavor powder is a clean label starch.

The resulting starch granules may have a porous structure on the surface and inside the granules. Preferably, they have a high number of large and small pores, which may or may not be connected to the hilum though internal channels.

In a preferred embodiment of the present invention, the porous starch has multiple pores on the surface with diameter comprised between 0.01 μm and 5 μm, preferably between 0.05 μm and 2.5 μm, and more preferably between 0.1 μm and 1 μm.

The porosity may be observed using scanning electron microscopy.

The particle size of the resulting starch granules may be further reduced by grinding, homogenizing or micronization before or after enzyme hydrolysis.

In a preferred embodiment of the present invention, the porous starch has a particle diameter comprised between 0.1 μm and 200 μm, preferably between 0.5 μm and 100 μm, and more preferably between 1 μm and 20 μm.

The particle diameter may be measured by laser diffraction particle sizer (Beckman Coulter LS 13 320).

In a preferred embodiment of the present invention, the porous starch used in the present invention is not gelatinized but is under granular form. During the process for fabricating the flavor powder, the porous starch will be gelatinized by heating.

In a preferred embodiment of the present invention, the flavor powder is obtained by a process comprising a heating step wherein the porous starch is gelatinized before or after being added to the flavoring solution, followed by a spray-drying step.

In a preferred embodiment of the present invention, the flavor powder is obtained by a process comprising the steps of:
(1) mixing a flavoring solution with a porous starch as defined in the present invention until obtaining a homogenous mixture,
(2) heating the mixture obtained in step (1) above the gelatinization temperature of the porous starch, and
(3) spray-drying the mixture obtained in step (2).

In a preferred embodiment of the present invention, the flavor powder is obtained by a process comprising the steps of:
(1) heating a porous starch as defined in the present invention above the gelatinization temperature of the porous starch,
(2) mixing a flavoring solution with the porous starch obtained in step (1) until obtaining a homogenous mixture, and
(3) spray-drying the mixture obtained in step (2).

As used herein, the expression “flavoring solution” refers to a solution, including extract, sauce, emulsion, and suspension, used to produce the flavor powder by drying, in particular spray-drying. In particular, “flavoring solution” refers to a seasoning sauce.

The flavoring solution of the present invention can be used for example in the flavorings or seasonings for noodles, rice, puffed foods, snacks, biscuits, beverages and soups.

In a preferred embodiment of the present invention, the flavoring solution is a bouillon (soup stock, broth, and meat extract), a seasoning sauce, a seed extract, a leaf or vegetable extract, a fruit extract, a mushroom extract, a yeast extract, a miso paste, a soy sauce, an artificial or synthetic flavor and mixtures thereof, and preferably a soy sauce.

As used herein, the expression “gelatinization” refers to the transition of porous starch from insoluble semi-crystalline granular structure to soluble amorphous non-granular structure, which takes place during the heating of the porous starch as defined in the present invention.

### Mixing Step

In a preferred embodiment of the present invention, a flavoring solution and the porous starch of the present invention are mixed by agitation or stirring at a temperature comprised between 0 and 50° C. for 1 minutes to 120 minutes, preferably between 15 and 35° C. for 10 minutes to 60 minutes and more preferably between 20 and 30° C. for 20 minutes to 40 minutes and even more preferably at approximately 25° C. for approximately 30 minutes. The mixing step may be stopped when the mixture reaches homogeneity, indicated by no starch sedimentation at the bottom of the container and when the mixture has stable and low viscosity. These characteristics can be visually observed by eyes. Water can be added to the mixture to reduce its viscosity.

### Heating Step

Dextrin and maltodextrin have higher solubility and hygroscopicity (higher content of simple sugars and smaller molecules without granular structure) than the porous starch of the present invention. Depending on the final application, it can be important that the flavor powder be soluble and that porous starch be gelatinized before spray drying. The porous starch may be gelatinized before or after being added to the flavoring solution. For puff snacks and biscuits, it is not important that the flavor powder be soluble. For noodles and soups, it is important that the flavor powder be completely soluble and thus that porous starch be gelatinized before spray drying, otherwise the starch will sediment at the bottom of the container.

Advantageously, the porous starch is gelatinized before the spray-drying step. This step of gelatinization will thus be called later “heating step”. The porous starch may be gelatinized before or after being added to the flavoring solution.

In the heating step, a high temperature is used to gelatinize the porous starch and destroy its semi-crystalline porous granular structure. As the result, the starch molecules become soluble. If the heating temperature is not high enough for a complete gelatinization, the porous starch might still have some residual granular structure. By contrast, if the heating temperature is too high, such as under high pressure system, it may cause the decomposition of the starch molecules, increasing the amount of simple sugars, causing the maillard reaction and/or caramelization, and producing burnt and off flavor.

Thus, in a preferred embodiment of the present invention, during the heating step the porous starch, before or after being added to the flavoring solution, is heated at a temperature comprised between 60 and 120° C. for 1 minute to 120 minutes, preferably between 75 and 100° C. for 15 minutes to 60 minutes and more preferably between 80 and 95° C. for 25 minutes to 40 minutes and even more preferably at approximately 90° C. for approximately 30 minutes.

The heating step can be combined with or considered as being a sterilization/pasteurization step and can be considered as a pre-treatment for spray drying. To ensure a food is safe for human consumption, it is mandatory to control the microbial counts of the food through a sterilization process or a pasteurization process. Sterilization refers to any process that causes a destruction of all microorganisms and their spores. One of the common sterilization processes is a heating step at high temperature, such as autoclave at 121° C. to 132° C. Pasteurization refers to any process that only kills pathogenic bacteria, which is a less harsh treatment than sterilization process. The temperature range of a pasteurization process is usually between 62° C. and 100° C. Thus, the porous starch before or after being added to the flavoring solution can be gelatinized during a sterilization process or a pasteurization process. In addition, because spray drying is performed at an inlet temperature comprised between 100 and 280° C., the heating step of the mixture of the flavoring solution and porous starch can be considered as a pre-treatment for spray drying, which reduces the time needed for the mixture to reach the spray-drying temperature.

In a preferred embodiment of the present invention, before the heating step the granular porous starch has a low viscosity comprised between 0.1 and 100 cP, preferably between 1 and 50 cP and even more preferably inferior to 25 cP at 30% starch concentration, 50° C., and stirring at 160 rpm.

By “at 30% starch concentration”, it is herein understood a system containing 30% by weight of dry starch and 70% by weight of water.

The viscosity of porous starch after gelatinization is very important. A customer usually uses more than 30% by weight of dry substance with respect to the total weight of the mixture for spray drying. If the viscosity is too high, the mixture solution for spray-drying cannot be pumped and sprayed through the nozzle of spray dryer. Advantageously, the viscosity of porous starch after gelatinization has to be similar to that of dextrin and maltodextrin at the same level of dry substance by weight with respect to the total weight of a mixture obtained by mixing a flavoring solution with dextrin or maltodextrin.

Advantageously, after the heating step, the porous starch of the present invention becomes soluble and has low viscosity similar to or better than that of dextrin and maltodextrin.

In a preferred embodiment of the present invention, after the heating step as previously defined the porous starch becomes soluble and has a viscosity comprised between 0.1 and 400 cP at 30% of starch concentration, 50° C. and stirring at 160 rpm, preferably between 1 and 250 cP and even more preferably inferior to 150 cP at 30% starch concentration, 50° C., and stirring at 160 rpm.

In the present invention, the viscosity may be measured using a Rapid Visco Analyser (RVA 4500, Perten Instruments).

The particle size of the porous starch before the heating step may be the same as that of their corresponding native starch, which varies with botanical origin. For good spray drying, the particle size should not be too big to avoid the blocking of the spray nozzle during spray-drying.

In a preferred embodiment, the porous starch before the heating step has a particle size inferior to 200 μm, preferably inferior to 100 μm, more preferably inferior to 20 μm, and even more preferably between 0.1 μm and 20 μm.

In a preferred embodiment, the porous starch after the heating step has a particle size inferior to 100 μm, preferably inferior to 50 μm, more preferably inferior to 10 μm, and even more preferably between 0.1 μm and 10 μm.

As previously explained, depending on the final application, it can be important that the flavor powder be completely soluble and thus that the porous starch be gelatinized before spray drying, otherwise the starch will sediment at the bottom of the container.

The porous starch before the heating step may be 100% insoluble in water.

In a preferred embodiment, before the heating step the amount of water-insoluble material in the mixture of flavoring solution and porous starch is comprised between 5% to 65%, preferably between 20% and 50% and more preferably between 30% and 45% by weight with respect to the total weight of the mixture of flavoring solution and porous starch.

In a preferred embodiment, after the heating step the amount of water-insoluble material in the porous starch is comprised between 0% to 70%, preferably between 0.1% and 50% and more preferably between 1% and 30% by weight with respect to the total weight of the porous starch.

In a preferred embodiment, after the heating step the amount of water-insoluble material in the mixture of flavoring solution and the porous starch is comprised between 0% to 50%, preferably between 0.1% and 30% and more preferably between 0.5% and 20% by weight with respect to the total weight of the mixture of flavoring solution and porous starch.

In the present invention, the amount of water-insoluble material is measured by centrifuging the solution or suspension, and then the precipitate is collected and oven-dried. The amount of water-insoluble material is calculated as the dry weight of precipitate divided by the initial weight of the solution or suspension, and expressed as weight percentage.

In a preferred embodiment of the present invention, after the heating step the porous starch does not have a semi-crystalline granular structure anymore. The starch becomes an amorphous and non-granular starch.

### Spray-Drying Step

Advantageously, the porous starch used in the spray-drying step has been gelatinized, has a low viscosity similar to that of dextrin and maltodextrin and does not have a semi-crystalline granular structure anymore.

In a preferred embodiment of the present invention, in step (3) the mixture obtained in step (2) is spray dried at an inlet temperature comprised between 100 and 280° C., preferably comprised between 120 and 220° C. and more preferably comprised between 130 and 180° C.

In a preferred embodiment of the present invention, in step (3) the mixture obtained in step (2) is spray dried at an outlet temperature comprised between 40 and 140° C., preferably comprised between 50 and 100° C. and more preferably comprised between 60 and 80° C.

The moisture content of spray-dried powder may be analyzed using a moisture analyzer (MA37-1CN, Sartorius). The resulting moisture content should be below 15%, preferably below 10%, and more preferably below 5% by weight with respect to the total weight of the spray-dried powder.

In the present invention, the porous starch as defined in the present invention is used in the preparation of a flavor powder to replace 100% of dextrin/maltodextrin in a flavor powder.

Advantageously, the porous starch is used a clean label replacement for maltodextrin and dextrin in a flavor powder.

In the present invention, the amount of spray drying aid added to the flavoring solution for the preparation of the flavor powder can be reduced as the porous starch is more effective than maltodextrin and dextrin, as the porous starch contains larger molecules and less mono- and disaccharides. Indeed, mono- and disaccharides are more hygroscopic than larger molecules, increasing the stickiness. In addition, larger molecules have higher Tg than small molecules, and therefore are more effective as spray drying aid.

In a preferred embodiment, the flavor powder comprises from 10% to 90%, preferably from 30% to 70% and even more preferably from 40% to 60% by weight of porous starch with respect to the total weight of the flavor powder.

In a preferred embodiment, the flavor powder comprises from 10% to 80%, preferably from 25% to 65% and even more preferably from 35% to 50% by weight of flavoring components, such as soy sauce solids, with respect to the total weight of the flavor powder.

In a preferred embodiment, the flavor powder comprises from 0% to 40%, preferably from 10% to 30% and even more preferably from 15% to 25% by weight of additives with respect to the total weight of the f flavor powder. The additives comprise, but not limited to, sodium chloride, monosodium glutamate, caramel color, and mixtures thereof.

In a preferred embodiment, the flavor powder comprises:
from 10% to 90%, preferably from 30% to 70% and even more preferably from 40% to 60% by weight of porous starch with respect to the total weight of the flavor powder,
from 10% to 80%, preferably from 25% to 65% and even more preferably from 35% to 50% by weight of flavoring components, such as soy sauce solids, with respect to the total weight of the flavor powder, and
from 0% to 40%, preferably from 10% to 30% and even more preferably from 15% to 25% by weight of additives with respect to the total weight of the flavor powder.

A second object of the present invention relates to a process of fabricating a flavor powder comprising a step of adding porous starch as defined in the present invention as spray-drying aid, wherein said process does not comprise a step of adding maltodextrin and/or dextrin.

In a preferred embodiment of the present invention, the process further comprises a mixing step, a heating step and a spray-drying step as defined in the present invention.

Thus, in a preferred embodiment of the present invention, the process comprises the steps of:
(1) mixing a flavoring solution with a porous starch as defined in the present invention until obtaining a homogenous mixture,
(2) heating the mixture obtained in step (1) above the gelatinization temperature of the porous starch, and
(3) spray-drying the mixture obtained in step (2), as defined in the present invention.

In another preferred embodiment of the present invention, the process comprises the steps of:
(1) heating a porous starch as defined in the present invention above the gelatinization temperature of the porous starch,
(2) mixing a flavoring solution with the porous starch obtained in step (1) until obtaining a homogenous mixture, and
(3) spray-drying the mixture obtained in step (2),
as defined in the present invention

In a preferred embodiment of the present invention, the dry powder obtained in step (3) is stored, and preferably is stored in a sealed bag, under dry conditions at room temperature.

A third object of the present invention relates to a flavor powder comprising porous starch as defined in the present invention obtained from the process as defined in the present invention.

A fourth object of the present invention relates to flavor powder comprising a spray-drying aid containing or consisting of porous starch as defined in the present invention, wherein said flavor powder does not contain dextrin or maltodextrin.

Thanks to the specific use of porous starch as spray-drying aid as defined in the present invention it is possible to provide a flavor powder which contains clean-labelled ingredients and does not contain dextrin and maltodextrin. Indeed, porous starch which is perceived as a natural and healthy ingredient by the consumers, is classified as a clean label ingredient and has a chemical structure and a viscosity similar to dextrin and maltodextrin. Furthermore, since the porous starch has a lower amount of simple/reducing sugars than dextrin and maltodextrin, the resulting flavor powder may have a less scorch odor, a less burnt flavor and a lighter color (less maillard reactions occur).

Advantageously, the porous starch is obtained from enzymatic hydrolysis and the process does not require the use of acid or bases except for adjusting the pH for enzyme hydrolysis and enzyme deactivation. Furthermore, the porous starch is not chemically substituted, oxidized, or reduced.

Thanks to the specific proprieties of porous starch, the mixture of the flavoring solution and porous starch can be spray-dried in order to obtain the corresponding flavor powder without difficulties. Advantageously, in the present invention, porous starch is mixed with flavoring solutions, solubilized and gelatinized by heating as part of the sterilization or pasteurization process prior to spray drying. The resulting solution has a low viscosity and improved solubility after gelatinization which is similar to that of a solution containing dextrin and maltodextrin and is thin enough for spray drying to produce the flavor powder.

The flavor powder of the present invention may have lower hygroscopicity and hence lower caking trend than that made with dextrin or maltodextrin, therefore the flavor powder of the present invention may have finer powder particles.

Furthermore, the flavor powder of the present invention may have less burnt flavor (less maillard reaction) and more intense flavor (less spray drying aid may be used) than that made with dextrin or maltodextrin. It may also have lighter color.

The invention will now be illustrated by means of the following figures and examples, it being understood that these are intended to explain the invention, and in no way to limit its scope.

### BRIEF DESCRIPTION OF THE DRAWINGS

FIG. 1: Comparison of Rapid Viscosity Analysis (RVA) pasting profile between native and porous rice starches at 10% solid content.

FIG. 2: Comparison of RVA pasting profile between native and porous maize starches at 10% solid content.

FIG. 3: Comparison of RVA pasting profile between native and porous tapioca starches at 10% solid content.

FIG. 4: RVA pasting profiles of porous starches at 30% solid content (A) and of 7-day stored starch paste (B).

FIG. 5: Scanning electron microscopic images of (A) native rice starch, (B) porous rice starch, (C) native maize starch, (D) porous maize starch, (E) native tapioca starch, and (F) porous tapioca starch.

FIG. 6: Solubility of soy sauce powders made using maltodextrin and porous rice starch at different temperature.

FIG. 7: Pasting profiles of soy sauce powders at 10% soy sauce suspension.

FIG. 8: Pasting profiles of soy sauce powders at 30% soy sauce suspension.

FIG. 9: Moisture sorption profiles of soy sauce powders at 30° C., 70% RH.

FIG. 10: Appearance of soy sauce powders after storage at 30° C., 70% RH.

FIG. 11: Pasting profiles of porous rice starch and porous waxy rice starch at 10% solid content.

FIG. 12: Pasting profiles of porous rice starch and porous waxy rice starch at 30% solid content.

### EXAMPLES

In the following examples, the following commercial products are used:
Rice starch from Jiangsu Baobao Suqian National Biotechnology Co., Ltd.,
Waxy rice starch is a sample from Anhui Shunxin Shengyuan Biological Food Co., Ltd.
Tapioca starch from Ubon Agricultural Energy Co., Ltd.,
Maize starch from Roquette,
Maltodextrin DE 12 (Glucidex® 12D) commercialized by Roquette,
NaOH from Sinopharm,
Liquozyme Supra 2.2× (alpha-amylase) from Novozymes, and
HCl from Sinopharm.

The native rice starch, native maize starch and native waxy rice starch used in examples 1 to 3 were produced according to the protocol mentioned in the first example of starch extraction process described in the description. Whereas, the native tapioca starch used in example 1 was produced according to the protocol mentioned in the second example of starch extraction process described in the description.

The porous rice starch, the porous tapioca starch, the porous maize starch, and the porous waxy rice starch used in examples 1 to 3 were produced according to the following protocol:
1) Preparing 35% of dry substance by weight of native starch slurry,
2) Stirring at 300 rpm/min and increasing the temperature to 50° C.,
3) Slowly adding 5% NaOH solution to adjust the pH to 6.5 and equilibrating the temperature to 50° C.,
4) Adding Liquozyme Supra 2.2× (alpha-amylase) and mixing completely (5 mg enzyme/g dry starch) for 3 hours at 50° C. in examples 1 and 2 or for 6 hours at 50° C. for example 3, 5) After 3 hours adding 5% HCl solution to decrease the pH to 3.5 and allowing it to react for 30 min while slowly cooling to room temperature with stirring,
6) After 30 min, adjusting the pH to 5.5 by adding 5% NaOH solution,
7) Filtering the sample by vacuum filtration and washing the starch by water twice, and
8) Drying the porous starch by oven at 45° C. until the moisture content be 12% or less.

The soy sauce powder of example 2 was produced according to the following protocol:
1) Mixing 2000 g Haitian soy sauce (Brix 28%) with 700 g of porous rice starch,
2) Heating the mixture at 80-85° C. for 30 min to create a solution with 45% by weight of dry substance with respect to the total weight of the mixture obtained in step (1). Starch is gelatinized in this stage.
3) Spray drying the mixture at inlet temperature of 170° C., outlet temperature of 70° C., and feeding speed of 28 mL/min using a Yamato spray dryer (ADL311).
4) Keeping the dry powder in a sealed bag under dry condition at room temperature.

### Example 1: Comparison of Properties of Porous Starches (Porous Rice Starch, Porous Tapioca Starch and Porous Maize Starch) and Maltodextrin DE 12

### Pasting Properties:

Each porous starch, native starch, and maltodextrin sample (2.5 g, dry weight basis) to analyze was mixed with water to a final total weight of 25 g (10% starch suspension or solid content) in an aluminum canister.

To emphasize the differences between porous starch and maltodextrin samples, a higher concentration at 30% solid content was also used where 7.5 g (dry weight basis) sample to analyze was mix with water to a final total weight of 25 g in the aluminum canister. The resulting starch and maltodextrin paste samples from RVA test were stored in a refrigerator for 7 days and retested using RVA with the same heating profile.

Then, each sample to analyze was heated using a Rapid Visco Analyser (RVA 4500, Perten Instruments) according to the heating profile presented in table 1 while measuring viscosity and pasting temperature.

TABLE 1

| Time | Temperature (° C.) | Shearing speed (rpm) |
|---|---|---|
| 00:00:00 | 50 | 960 |
| 00:00:10 | 50 | 160 |
| 00:01:00 | 50 | 160 |
| 00:04:45 | 95 | 160 |
| 00:07:15 | 95 | 160 |
| 00:11:00 | 50 | 160 |
| 00:13:00 | 50 | 160 |

Pasting temperature is the temperature at which the viscosity starts to increase, identified by viscosity increase by more than 24 cP within 0.1 min.

Peak viscosity is the highest viscosity during heating and holding at 95° C., trough is the lowest viscosity during holding at 95° C., final viscosity is the highest viscosity during cooling and holding at 50° C., breakdown is the difference between peak viscosity and trough, and setback is the difference between final viscosity and trough.

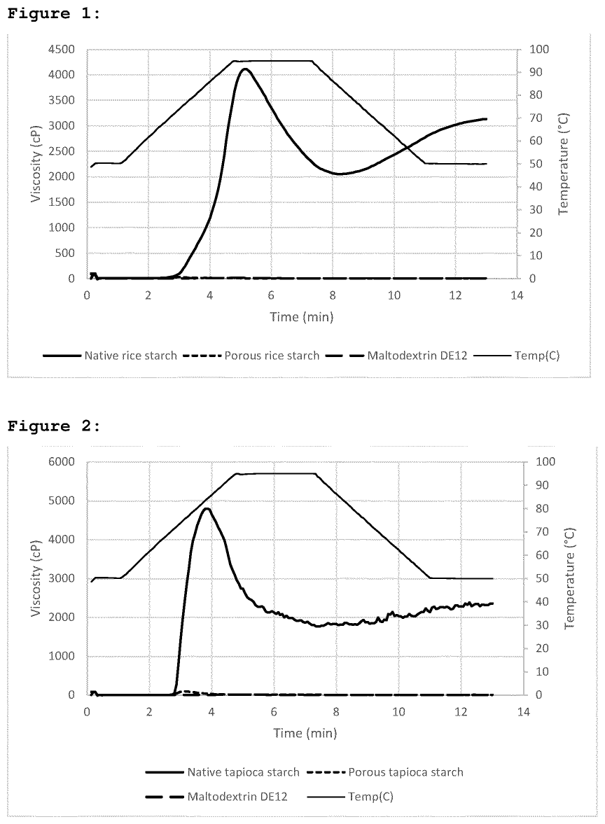

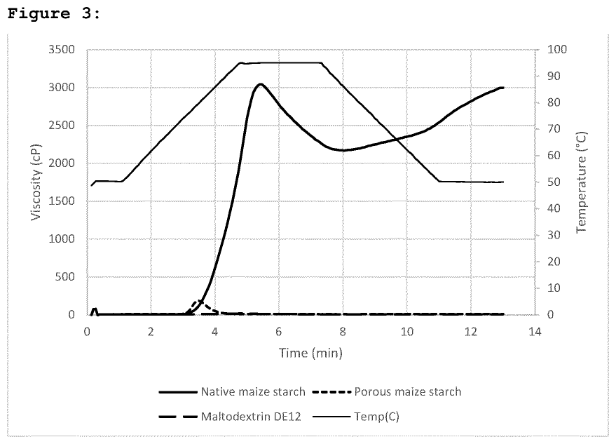

Results are shown on FIGS. 1 to 4.

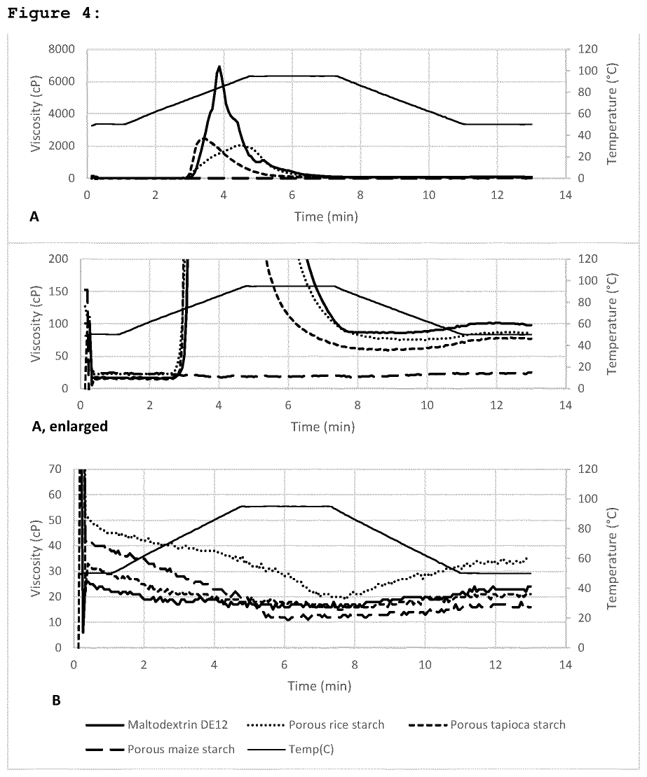

As shown on FIGS. 1 to 3, the viscosities of rice, tapioca, and maize starches were substantially reduced after the hydrolysis using Liquozyme Supra 2.2× (alpha-amylase) (see the viscosity of porous starches in comparison to their corresponding native starches). The peak viscosities of native starches at 10% solid content were equal or higher than 3000 cP, whereas those of porous starches were lower than 200 cP. The final viscosities of native starches at 10% solid content were above 2000 cP, whereas those of porous s starches were lower than 20 cP. The viscosity differences between porous starches and maltodextrin DE 12, however, were not obvious at 10% solid content, where all final viscosities were lower than 20 cP (see FIGS. 1 to 3). Therefore, a higher concentration (30% solid content) was used to emphasize the differences between the viscosities of porous starches and maltodextrin DE 12 (see FIG. 4A). All porous starches showed a peak viscosity during heating, related to the gelatinization of the porous starch, which decreased rapidly upon further heating and stirring, due to the disintegration of the granular structure. This phenomenon was not observed from maltodextrin, which was devoid of granular structure. The maltodextrin also maintained its viscosity throughout the RVA analysis between 15 and 25 cP. The peak viscosities of porous starches at 30% solid content were between 2000 and 7000 cP, where the porous rice starch had the lowest peak viscosity. The final viscosities of the porous starches were between 50 to 100 cP, where the porous maize starch had the lowest final viscosity. At 10% solid content, the viscosity of porous starch and maltodextrin is too low for RVA detection. In addition, at 30% solid content there is less water which will affect the swelling of the starch granules.

After 7 day cold storage, all porous starches and maltodextrin at 30% solid content did not show peak viscosity because they were all devoid of granular structure (see FIG. 4B). Their viscosities ranged between 10 and 50 cP, which were very small for RVA detection. Maltodextrin DE 12 had the lowest initial viscosity, whereas porous maize starch had the lowest final viscosity.

### Gelatinization/Thermal Properties:

Gelatinization properties of each sample were measured by Differential Scanning calorimetry (DSC 1, Mettler Toledo) according to the following protocol.

Each starch sample (2-3 mg, dry weight basis) to analyze was mixed with water at a weight ratio of starch to water of 1:3. The mixture was sealed in a standard 40 μL aluminum pan and allowed to equilibrate for at least an hour. The pan was then equilibrated again in the DSC at 10° C. for 1 min followed by heating to 100° C. at 10° C./min.

Onset temperature (Tₒ), peak temperature (Tₚ), end temperature (T_{c}) and enthalpy change were obtained using the software provided by Mettler Toledo (STARe system).

The enthalpy change of starch gelatinization was obtained based on the area under the curve. After the gelatinization test, the pans were stored in a refrigerator for 15 days and reanalyzed using the same heating condition to obtain the retrogradation properties of starch samples based on the endotherm related to the melting of the retrograded starch. The rate of retrogradation is the enthalpy change of the melting of retrograded starch divided by the enthalpy change of starch gelatinization.

Results are shown in table 2 below.

TABLE 2
Thermal properties of native and porous starches

| | Gelatinization | | | | Melting of retrograded starch | | | | Rate of |
|---|---|---|---|---|---|---|---|---|---|
| | Onset | Peak | Endset | | Onset | Peak | Endset | | retro- |
| | temperature | temperature | temperature | ΔH | temperature | temperature | temperature | ΔH | gradation |
| Sample | (° C.) | (° C.) | (° C.) | (J/g) | (° C.) | (° C.) | (° C.) | (J/g) | (%) |
| Native | 59.77 ± | 65.26 ± | 82.46 ± | 12.70 ± | 44.13 ± | 53.10 ± | 60.30 ± | 4.54 ± | 35.84% ± |
| rice | 0.34 | 0.28 | 0.81 | 1.38 | 1.47 | 0.80 | 0.40 | 0.47 | 2.41% |
| Porous | 62.31 ± | 66.61 ± | 85.03 ± | 11.16 ± | ND | ND | ND | ND | ND |
| rice | 0.10 | 0.11 | 0.95 | 0.67 | | | | | |
| Native | 65.33 ± | 70.70 ± | 74.97 ± | 12.22 ± | 42.67 ± | 52.72 ± | 60.72 ± | 6.70± | 54.81% ± |
| maize | 0.69 | 0.27 | 0.57 | 0.15 | 2.85 | 1.91 | 0.54 | 0.41 | 2.68% |
| Porous | 65.83 ± | 71.05 ± | 75.25 ± | 10.84 ± | ND | ND | ND | ND | ND |
| maize | 0.06 | 0.00 | 0.13 | 1.35 | | | | | |
| Native | 63.77 ± | 68.62 ± | 76.24 ± | 14.92 ± | 42.74 ± | 54.39 ± | 61.22 ± | 5.79 ± | 38.88% ± |
| tapioca | 0.23 | 0.35 | 0.46 | 0.48 | 1.56 | 1.17 | 0.90 | 0.51 | 4.67% |
| Porous | 65.27 ± | 69.01 ± | 76.07 ± | 13.30 ± | 44.87 ± | 54.12 ± | 64.07 ± | 2.38 ± | 17.75% ± |
| tapioca | 0.35 | 0.50 | 0.28 | 0.88 | 0.35 | 1.07 | 3.22 | 0.74 | 4.39% |

*ND = not detected

As shown in table 2, the onset temperature, peak temperature and endset temperature of native starches slightly increased after enzyme treatment (see the onset temperature, peak temperature and endset temperature of native starches in comparison to their corresponding porous starches). This was due to the annealing effect during enzyme treatment.

Annealing is the rearrangement of starch crystalline structure which takes place when the starch granules are heated in excess water below the gelatinization temperature. The enzyme reaction temperature 50° C. can act as an annealing temperature. Furthermore, the small molecules in the porous starches as the result of enzyme hydrolysis have higher mobility than the molecules in the native counterparts.

Therefore, the porous starch samples showed higher gelatinization temperature than their native counterparts as the result of annealing.

There was no detectable melting peak of porous rice and porous maize starches stored in a refrigerator for 15 days after gelatinization, indicating that the external branches of starch molecules from these porous starches were too short for retrogradation. Retrogradation is the recrystallization of starch molecules where the external branches reform into double helices and align themselves into repeating crystalline structure. Therefore a certain length of external branches is needed to effectively form double helices. After enzyme hydrolysis, the external branches of porous rice and porous maize starches might be too short to retrograde. On the other hand, a lower rate of retrogradation was observed for porous tapioca starch as compared with its native counterpart. It is well known that tuber and root starches have longer external branches than most cereal starches, and it seemed that after enzyme hydrolysis there was still substantial length of external branches in porous tapioca starch to retrograde upon cold storage.

Lower rate of retrogradation of porous starch is an additional benefits compared with native counterparts. It indicates that the porous starch is stable in solution form during storage, especially at cold temperature. For example, the solution containing porous starch will remain the same appearance (no increase in haziness or cloudiness) and viscosity during cold storage.

### Scanning Electron Microscopy:

Starch granules were mounted directly onto aluminum stubs using double-sided adhesive tape, and then coated with 20 nm gold under vacuum. Images of starch granules were obtained with a field emission SEM (EVO 18, Zeiss) at an acceleration potential of 10 kV and magnification of ×2000.

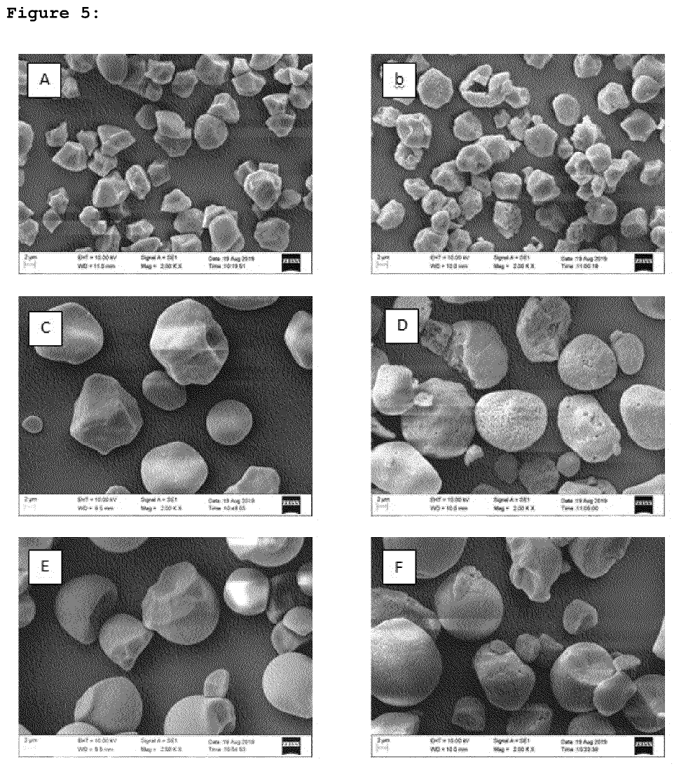

Results are shown on FIG. 5.

As shown on FIG. 5, rice starches had the smallest granular size among the three starches. Rice and maize starches had polygonal shape, whereas tapioca starch had dome shape. In general, all native starches had smooth surface structure, whereas porous starches showed granular structure with tiny pores on the surface and broken granules.

### Particle Size Analysis:

The particle size of porous starch was analyzed by laser diffraction particle sizer (Beckman Coulter LS 13 320).

Results are shown in table 3 below.

TABLE 3
Particle size of native and porous starches.

| | | | Mean/Median | | | | | | | | | |
|---|---|---|---|---|---|---|---|---|---|---|---|---|
| Sample | Mean | Median | ratio | Modus | D₁₀ | D₅₀ | D₉₀ | >20 um | >40 um | >75 um | >100 um | >150 um |
| Native | 14.9 | 8.85 | 1.683616 | 7.775 | 4.542 | 8.85 | 37.66 | 20.9% | 8.76% | 0.28% | 0% | 0% |
| rice | | | | | | | | | | | | |
| Porous | 8.98 | 6.353 | 1.413505 | 5.878 | 3.737 | 6.353 | 13.19 | 9.39% | 2.23% | 0% | 0% | 0% |
| rice | | | | | | | | | | | | |
| Native | 35.9 | 24.91 | 1.441188 | 18 | 10.41 | 24.91 | 75.58 | 60.4% | 31.1% | 10.2% | 4.33% | 0.95% |
| maize | | | | | | | | | | | | |
| Porous | 12.09 | 11.87 | 1.018534 | 13.61 | 5.856 | 11.87 | 18.62 | 5.98% | 0% | 0% | 0% | 0% |
| maize | | | | | | | | | | | | |
| Native | 24.1 | 20.63 | 1.168202 | 19.76 | 9.844 | 20.63 | 42.52 | 52.3% | 12.2 | 1.09% | 0.045% | 0% |
| tapioca | | | | | | | | | | | | |
| Porous | 13.48 | 13.37 | 1.008227 | 14.94 | 7.23 | 13.37 | 20.02 | 10.1% | 0% | 0% | 0% | 0% |
| tapioca | | | | | | | | | | | | |

All porous starches had smaller particle sizes than their native counterparts. This could be due to the broken granules as seen in the SEM images on FIG. 5.

### X-Ray Diffractiometry:

X-ray diffactogram patterns of different samples were obtained with a D/Max-2200 X-ray diffractometer (Rigaku Denki Co.) using Cu Ka radiation at 44 kV and 26 mA. The samples were scanned in the range of 4-45° (2θ) at the rate of 5°/min. Relative crystallinity was calculated by the ratio of the crystalline area to the total diffractogram area.

Results are shown in table 4 below.

| Sample | Crystalline pattern | Relative crystallinity (%) |
|---|---|---|
| Native rice | A | 10.7 |
| Porous rice | A | 11.9 |
| Native maize | A | 12.3 |
| Porous maize | A | 12.4 |
| Native tapioca | A | 13.3 |
| Porous tapioca | A | 15.3 |

### Table 4—Crystalline Pattern and Relative Crystallinity of Native and Porous Starches.

As shown in table 4, all samples had the A-type crystalline pattern (peaks at 2θ of 15, 17, 18, and 23°). The porous starches had slightly higher relative crystallinity than their native counterpart, probably due to the crystalline part is more resistant to enzyme hydrolysis than the amorphous part.

### CONCLUSION

The viscosities of rice, tapioca, and maize starches were substantially reduced after the hydrolysis using Liquozyme Supra 2.2× (alpha-amylase). Although it is not obvious at 10% solid content, the viscosities of porous starches were still a little higher than that of maltodextrin with DE 12 at 30% solid content. However, after 7 day cold storage, all porous starch and maltodextrin DE 12 samples showed similar paste viscosities at 30% solid content. The onset temperature, peak temperature and endset temperature of the three starches increased after enzyme treatment. There was no retrogradation for porous rice and porous maize starches after stored in a refrigerator for 15 days after gelatinization, and a lower rate of retrogradation was observed for porous tapioca starches compared with its native counterpart. All porous starches had smaller particle size, slightly higher relative crystallinity, and more tiny pores on the granule surface than their native counterparts.

### Example 2: Comparison of Soy Sauce Powders Made with Maltodextrin DE 12 and Porous Rice Starch

### Solubility:

The solubility of the soy sauce powder was measured according to the following protocol.
1. Placing 200 mg soy sauce powder in a 15-mL centrifuge tube.
2. Adding RO water to reach 10 g total weight and mix well.
3. Placing the sample in a water bath at 30° C., 50° C., 60° C., 70° C., or 80° C. with occasional shaking for 30 mins.
4. After cooling to room temperature, centrifuge at 4,000 rpm for 10 min.
5. Pouring the supernatant into a tared weighing bottle.
6. Drying the supernatant in an oven at 110° C. overnight and weigh the dry weight.

Soluble=dry weight of supernatant/dry weight of soy sauce powder*100%

Results are shown on FIG. 6.

As shown on FIG. 6, the soy sauce powder made with maltodextrin had high solubility, which reached about 94% at 30° C. (close to ambient temperature). The soy sauce powder made with porous starch had lower solubility. The solubility was inferior to 70% at 30° C. It increased to 78% and 82% after being heated at 70° C. and 80° C., respectively. The solubility of the soy sauce powder can be improved by heating the soy sauce-porous starch mixture at higher temperature before spray drying.

### Pasting Properties/Viscosity:

The viscosity of soy sauce powder was analyzed using Rapid Viscosity Analysis (RVA) at 10% and 30% of soy sauce suspension. Soy sauce powder (2.5 g or 7.5 g, dry weight basis) was mixed with water to a final total weight of 25 g (10% and 30% soy sauce suspensions, respectively) in an aluminum canister. The heating profile is presented in table 1 of example 1.

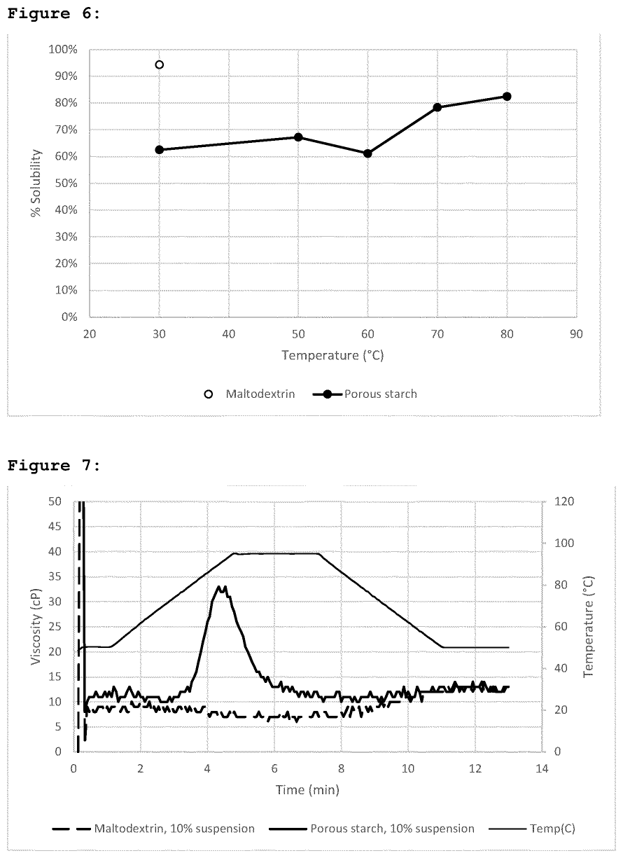

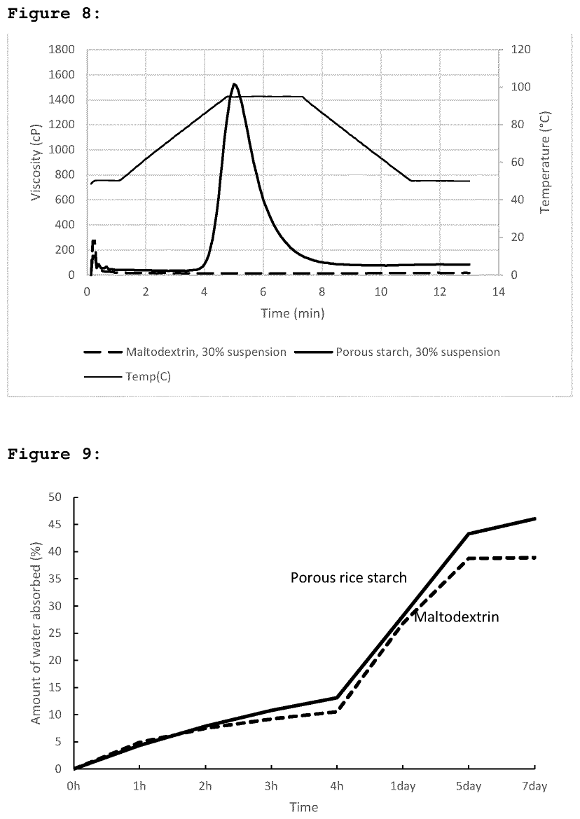

Results are shown on FIGS. 7 and 8 and on table 5 below.

TABLE 5
The pasting properties of soy sauce powders at 10% and 30% soy sauce suspensions.

| | | Pasting | Peak | Trough | | Final | |
|---|---|---|---|---|---|---|---|
| | Solid | Temperature | Viscosity | Viscosity | Breakdown | Viscosity | Setback |
| Sample | content | (° C.) | (cP) | (cP) | (cP) | (cP) | (cP) |
| Maltodextrin | 10% | ND | 10 | 6 | 4 | 13 | 7 |
| Porous starch | 10% | ND | 33 | 10 | 23 | 13 | 3 |
| Maltodextrin | 30% | ND | 22 | 12 | 10 | 18 | 6 |
| Porous starch | 30% | 85 | 1525 | 77 | 1448 | 85 | 8 |

As shown on FIG. 7, at 10% by weight of soy sauce suspension, which than the actual is slightly higher concentration for seasonings in soup, the difference in term of viscosity between the two soy sauce powders made from maltodextrin and porous rice starch was not big (peak viscosity during heating 10 cP versus 33 cP, respectively). The final viscosities at 50° C. of the two soy sauce powders were similar at 13 cP.

The difference became obvious when the suspension percentage was increased to 30% (FIG. 8), with the soy sauce powder made with porous starch showing a peak viscosity, indicating that there was some ungelatinized starch in the soy sauce powder sample, which was not observed from the soy sauce powder made with maltodextrin. This can be avoided by making sure that all porous starch had been gelatinized prior to the spray drying step, such as heating at higher temperature. The final viscosity was slightly higher for the soy sauce powder made with porous rice starch than that made with maltodextrin (85 cP vs 20 cP). However, it needs to be borne in mind that 30% of suspension is a very high concentration for soy sauce powder in soup.

### Moisture Sorption:

The Moisture sorption of the soy sauce powder was measured according to the following protocol.
1. Weighing 10 g samples in the culture dish and record the weight
2. Placing at 30° C., 70% relative humidity (RH), take photos and weigh after 1 h, 2 h, 3 h, 4 h, 1 day, 5 days, and 7 days.

Amount of water absorbed(%)=(Weight after storage−initial weight)/Initial weight*100%

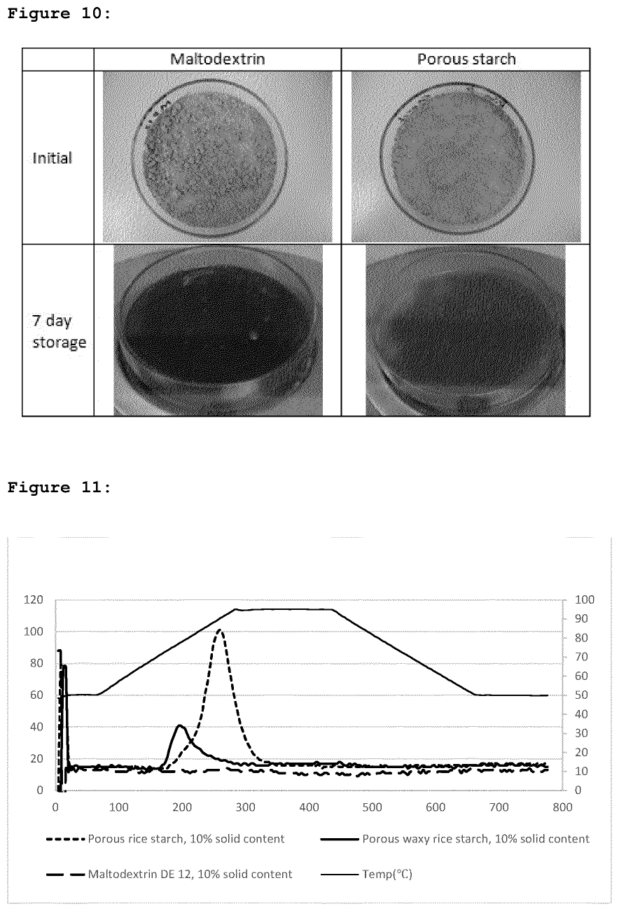

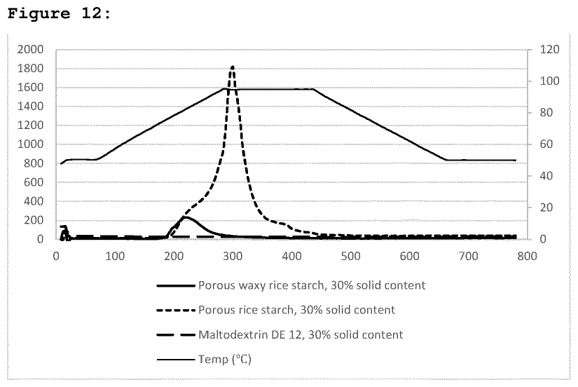

Results are shown on FIGS. 10 and 11.

As shown on FIG. 10, both soy sauces had similar water sorption profiles. As shown on FIG. 11, soy sauce powder made with porous starch at the initial state had a less caking trend (finer powder) than soy sauce powder made with maltodextrin DE12. Furthermore, the wetted soy sauce powder made with porous starch had a lighter color than the corresponding soy sauce powder made with maltodextrin DE 12 after 7 days of storage at 30° C., 70% RH although the other appearance was similar.

### CONCLUSION

The soy sauce powder made with porous rice starch had lower solubility than that made with maltodextrin at 30° C. (62% versus 94%). The solubility of the soy sauce powder made with porous rice starch increased to 78% and 82% after being heated at 70° C. and 80° C., respectively. At 10% by weight of soy sauce suspension, the difference in viscosity between the two soy sauce powders was quite small. The difference became obvious when the suspension percentage was increased to 30%, with the porous starch showing a peak viscosity, indicating that there was some ungelatinized starch in the soy sauce powder made with porous rice starch, which can be avoided by heating soy sauce-porous s starch mixture at higher temperature prior to spray drying. The final viscosity at 30% of suspension was slightly higher for the soy sauce powder made with porous rice starch than that made with maltodextrin, however it needs to be borne in mind that 30% of suspension is a very high concentration for soy sauce powder in soup. Both soy sauces had similar water sorption profiles. Soy sauce powder made with porous starch had a less caking trend (finer powder) than soy sauce powder made with maltodextrin DE 12. Furthermore, the wetted soy sauce powder made with porous starch had a lighter color than the corresponding soy sauce powder made with maltodextrin DE 12 after 7 days of storage at 30° C., 70% RH although the other appearance was similar.

The results showed that the soy sauce powder made with gelatinized porous starch behaved similar to that made with maltodextrin. The viscosity of the solution for spray drying and the powder is the most important. The solubility of soy sauce powder should be above 50%.

### Example 3: Comparison of Properties of Porous Rice Starch, Porous Waxy Rice Starch and Maltodextrin DE 12

### Pasting Properties/Viscosity:

The viscosity of porous rice starch and porous waxy rice starch was analyzed using Rapid Viscosity Analysis (RVA) at 10% and 30% of starch suspension and maltodextrin suspension. Porous starch or maltodextrin (2.5 g or 7.5 g, dry weight basis) was mixed with water to a final total weight of 25 g (10% and 30% solid content, respectively) in an aluminum canister. The heating profile is presented d in table 1 of example 1.

Results are shown on FIGS. 11 and 12.

As shown on FIG. 11, at 10% by weight of solid content, porous waxy rice starch had lower peak viscosity and lower pasting temperature than porous rice starch, that porous waxy rice starch is easily to be gelatinized prior to spray drying step. The final viscosities of porous rice starch and porous waxy rice starch were similar, which was lower than 20 cP and was similar to the viscosity of maltodextrin DE 12.

At 30% solid content, the difference between the two porous starches became more obvious (FIG. 12), with the porous rice starch showing much higher peak viscosity and much higher peak temperature (temperature at where the peak viscosity occurs) than the porous waxy rice starch, confirming that the porous waxy rice starch is more effective as spray drying aid as it is more easily gelatinized at lower water content. The final viscosity of porous waxy rice starch (at about 15 cP) was lower than that of maltodextrin DE 12 (at about 35 cP), whereas the final viscosity of porous rice starch was the highest, at about 40 cP.

1. A use of porous starch as spray-drying aid in the preparation of a flavor powder containing no maltodextrin and no dextrin.

2. The use according to claim 1, wherein the porous starch is selected from the group consisting of porous wheat starch, porous waxy wheat starch, porous maize starch, porous waxy maize starch, porous rice starch, porous waxy rice starch, porous tapioca starch, porous waxy tapioca starch, and mixtures thereof, and preferably porous rice starch and porous waxy rice starch.

3. The use according to claim 1, wherein the flavor powder is selected from a bouillon powder, a seasoning powder, a seed extract powder, a leaf or vegetable extract powder, a fruit extract powder, a mushroom extract powder, a yeast extract powder, a miso powder, a soy sauce powder an artificial or synthetic flavor powder and mixtures thereof, and preferably a soy sauce powder.

4. The use according to claim 1, wherein the porous starch is obtained from native starch granules by enzyme hydrolysis only and preferably by enzyme hydrolysis using alpha-amylase.

5. The use according to claim 1, wherein the flavor powder is obtained by a process comprising a heating step wherein the porous starch is gelatinized before or after being added to the flavoring solution, followed by a spray drying step.

6. The use according to claim 1, wherein the flavor powder comprises from 10% to 90%, preferably from 30% to 70% and even more preferably from 40% to 60% by weight of porous starch with respect to the total weight of the flavor powder.

7. The use according to claim 1, wherein the flavor powder comprises:
from 10% to 90%, preferably from 30% to 70% and even more preferably from 40% to 60% by weight of porous starch with respect to the total weight of the flavor powder,
from 10% to 80%, preferably from 25% to 65% and even more preferably from 35% to 50% by weight of flavoring components, such as soy sauce solids, with respect to the total weight of the flavor powder, and
from 0% to 40%, preferably from 10% to 30% and even more preferably from 15% to 25% by weight of additives with respect to the total weight of the flavor powder.

8. A process of fabricating a flavor powder comprising a step of adding porous starch as spray-drying aid, wherein said process does not comprise a step of adding maltodextrin and/or dextrin.

9. The process of fabricating a flavor powder according to claim 8, wherein the porous starch is selected from the group consisting of porous wheat starch, porous waxy wheat starch, porous maize starch, porous waxy maize starch, porous rice starch, porous waxy rice starch, porous tapioca starch, porous waxy tapioca starch, and mixtures thereof, and preferably porous rice starch and porous waxy rice starch.

10. The process of fabricating a flavor powder according to claim 8, wherein the flavor powder is selected from a bouillon powder, a seasoning powder, a seed extract powder, a leaf or vegetable extract powder, a fruit extract powder, a mushroom extract powder, a yeast extract powder, a miso powder, a soy sauce powder, an artificial or synthetic flavor powder and mixtures thereof, and preferably a soy sauce powder.

11. The process of fabricating a flavor powder according to claim 8, wherein the porous starch is obtained from native starch granules by enzyme hydrolysis only and preferably by enzyme hydrolysis using alpha-amylase.

12. The process of fabricating a flavor powder according to claim 8, wherein the flavor powder is obtained by a process comprising a heating step wherein the porous starch is gelatinized before or after being added to the flavoring solution, followed by a spray-drying step.

13. The process of fabricating a flavor powder according to claim 8, wherein the flavor powder comprises from 10% to 90%, preferably from 30% to 70% and even more preferably from 40% to 60% by weight of porous starch with respect to the total weight of the flavor powder.

14. The process of fabricating a flavor powder according to claim 8, wherein the flavor powder comprises:
from 10% to 90%, preferably from 30% to 70% and even more preferably from 40% to 60% by weight of porous starch with respect to the total weight of the flavor powder,
from 10% to 80%, preferably from 25% to 65% and even more preferably from 35% to 50% by weight of flavoring components, such as soy sauce solids, with respect to the total weight of the flavor powder, and
from 0% to 40%, preferably from 10% to 30% and even more preferably from 15% to 25% by weight of additives with respect to the total weight of the flavor powder.

15. The process of fabricating the flavor powder according to claim 8, wherein said process comprises the steps of:
(1) mixing a flavoring solution with a porous starch until obtaining a homogenous mixture,
(2) heating the mixture obtained in step (1) above the gelatinization temperature of the porous starch, and
(3) spray-drying the mixture obtained in step (2).

16. The process of fabricating the flavor powder according to claim 8, wherein said process comprises the steps of:
(1) heating a porous starch above the gelatinization temperature of the porous starch,
(2) mixing a flavoring solution with the porous starch obtained in step (1) until obtaining a homogenous mixture, and
(3) spray-drying the mixture obtained in step (2).

17. A flavor powder comprising porous starch obtained from the process as defined in claim 8.

18. A flavor powder comprising a spray-drying aid containing or consisting of porous starch, wherein said flavor powder does not contain dextrin or maltodextrin.

19. The flavor powder according to claim 18, wherein the porous starch is selected from the group consisting of porous wheat starch, porous waxy wheat starch, porous maize starch, porous waxy maize starch, porous rice starch, porous waxy rice starch, porous tapioca starch, porous waxy tapioca starch, and mixtures thereof, and preferably porous rice starch and porous waxy rice starch.

20. The flavor powder according to claim 18, wherein the flavor powder is selected from a bouillon powder, a seasoning powder, a seed extract powder, a leaf or vegetable extract powder, a fruit extract powder, a mushroom extract powder, a yeast extract powder, a miso powder, a soy sauce powder, an artificial or synthetic flavor powder and mixtures thereof, and preferably a soy sauce powder.

21. The flavor powder according to claim 18, wherein the porous starch is obtained from native starch granules by enzyme hydrolysis only and preferably by enzyme hydrolysis using alpha-amylase.

22. The flavor powder according to claim 18, wherein the flavor powder is obtained by a process comprising a heating step wherein the porous starch is gelatinized before or after being added to the flavoring solution, followed by a spray-drying step.

23. The flavor powder according to claim 18, wherein said flavor powder comprises from 10% to 90%, preferably from 30% to 70% and even more preferably from 40% to 60% by weight of porous starch with respect to the total weight of the flavor powder.

24. The flavor powder according to claim 18, wherein said flavor powder comprises:
from 10% to 90%, preferably from 30% to 70% and even more preferably from 40% to 60% by weight of porous starch with respect to the total weight of the flavor powder,
from 10% to 80%, preferably from 25% to 65% and even more preferably from 35% to 50% by weight of flavoring components, such as soy sauce solids, with respect to the total weight of the flavor powder, and from 0% to 40% preferably from 10% to 30% and even more preferably from 15% to 25% by weight of additives with respect to the total weight of the flavor powder.
